(19) United States
(12) Patent Application Publication
(10) Pub. No.: US 2025/0307436 A1
(43) Pub. Date: Oct. 2, 2025

(54) SYSTEM AND METHOD FOR SECURE KNOWLEDGE EXCHANGE AND PRIVACY-PRESERVING VALIDATION IN DECENTRALIZED AI APPLICATIONS

(71) Applicants: Michael David Doyle, Wheaton, IL (US); Yuxin Ruan, New York, NY (US); Paul F. Doyle, Grand Rapids, MI (US)

(72) Inventors: Michael David Doyle, Wheaton, IL (US); Yuxin Ruan, New York, NY (US); Paul F. Doyle, Grand Rapids, MI (US)

(21) Appl. No.: 19/097,609

(22) Filed: Apr. 1, 2025

Related U.S. Application Data
(60) Provisional application No. 63/572,907, filed on Apr. 1, 2024.

(51) Int. Cl. G06F 21/60; G06Q 20/38

(52) U.S. Cl. CPC G06F 21/606; G06Q 20/389

(57) ABSTRACT

A decentralized artificial intelligence platform is disclosed that enables secure sharing and execution of AI modules in sensitive application domains. The platform comprises a knowledge module registry for discovering specialized AI modules, a security framework enforcing structural integrity of AI modules, a zero-knowledge proof validation service for verifying usage conditions without exposing private data, and a blockchain-based value chain for managing smart contracts and payments. In operation, an AI agent can fetch a required AI module from the knowledge module registry, prove the right to use it, execute the AI module on local data, and automatically pay the AI module's provider through a blockchain transaction.

### CROSS-REFERENCE TO RELATED APPLICATION

This application claims the benefit of and priority to U.S. Provisional Application 63/572,907 filed Apr. 1, 2024, the disclosure of which is hereby incorporated by reference in its entirety as though fully set forth herein.

### TECHNICAL FIELD

The present subject matter relates generally to a decentralized artificial intelligence (AI) system.

### BACKGROUND

In conventional AI paradigms, large models and services are typically centralized and monolithic, which poses significant challenges for critical applications requiring stringent data protection. Key issues with existing centralized AI approaches can include data privacy and security risks, lack of specialization and IP vulnerability, opacity and trust issues, data localization and compliance challenges, and scalability and customization constraints. Regarding data privacy and security risks, centralized AI modules (e.g., large language models running on remote servers) concentrate sensitive data in one place, increasing the risk of breaches and unauthorized access. For example, patient health records sent to a central server may inadvertently expose private information, violating regulations like HIPAA or GDPR if the centralized system is compromised. Regarding lack of specialization and IP vulnerability, monolithic AI models follow a one-size-fits-all design and often lack the specialization required for niche tasks involving sensitive data. Customizing these general models for specific needs (such as a specialized medical diagnostic) usually requires inputting proprietary or confidential data for training, which raises intellectual property concerns. Once such data or custom model parameters are uploaded to a centralized service, the original owner loses control over how that information is stored or used, risking exposure or misuse of valuable proprietary knowledge. Regarding opacity and trust issues, centralized AI services often operate as “black boxes” with little transparency. Users and organizations have limited visibility into how their data is processed or safeguarded in these systems. This lack of transparency makes it difficult to assess or trust the system's handling of sensitive inputs, undermining confidence that private data will remain confidential and not be repurposed in unintended ways. Regarding data localization and compliance challenges, many jurisdictions have strict data localization laws requiring personal data to remain within certain geographic boundaries. Centralized AI platforms may not easily accommodate such requirements. For instance, a healthcare provider operating across different countries may struggle to use a single centralized AI service while complying with each region's privacy regulations, since centralized services typically store and process data in fixed locations that may not align with local laws. Regarding scalability and customization constraints, updating or scaling a monolithic, centralized AI model to serve new tasks or user-specific requirements can be cumbersome and inefficient. Any change to the central model potentially affects tasks (e.g., a custom model for a rare genetic test) without extensive retraining or duplication of the entire system. This all-or-nothing approach hinders innovation and prevents quick adaptation of AI capabilities to meet individualized needs

These challenges highlight the need for a new AI paradigm that can provide robust security and privacy guarantees while allowing modular customization and distributed deployment. There is a demand for an AI platform that moves away from centralization towards a decentralized architecture in which data stays local, specialized AI modules can be seamlessly integrated, and trust is established through transparent and verifiable means. Such a platform would ideally ensure that sensitive information (like personal medical or financial data) remains protected, that domain-specific AI expertise can be plugged in without exposing intellectual property, and that all transactions or usage of AI modules are traceable and enforceable in a transparent manner. The disclosed decentralized AI platform (referred to herein as “DecenAI”) addresses the aforementioned needs by incorporating structural integrity enforcement, privacy-preserving validation techniques, and a blockchain-based value exchange mechanism.

### BRIEF DESCRIPTION OF FIGURES

FIG. 1 is a block diagram illustrating an exemplary architecture of a decentralized AI platform in a healthcare in accordance with various aspects described herein.

FIG. 2 is a block diagram illustrating an exemplary method of a using the decentralized AI platform of FIG. 1, in accordance with various aspects described herein.

### DETAILED DESCRIPTION

Aspects of the disclosure described herein are directed to a comprehensive decentralized AI (DecenAI) platform that combines a standardized modular AI framework, a robust module registry, a zero-knowledge proof-based validation mechanism, and a value chain ledger for governance of usage and payments. The DecenAI platform described herein can be implemented in various forms of distributed computing environments. In a non-limiting example, the DecenAI platform comprises a network of computing nodes (such as servers, personal computers, or specialized hardware devices) that communicate over public or private networks (e.g., the Internet). Some of these nodes may operate as AI agents for end users or organizations while others provide infrastructure services like the module registry, zero-knowledge proof validation mechanism, or the value chain ledger.

It should be understood that the functional components described (registry, validation service, ledger, etc.) can be co-located or distributed across multiple physical machines, and the term “platform” generally refers to the collection of these functionalities globally accessible to participants in the system.

The term “AI agent” as used herein refers to any computing or software entity capable of autonomous action in the platform (including software agents and processes acting on behalf of users or providers). In a non-limiting example, an AI agent can be an application running on a doctor's computer or a hospital server, configured with the credentials and permissions of that doctor, such that it can request AI modules, handle patient data input, and return results. Similarly, a service provider's AI agent could be a cloud service managing one or more AI modules and interfacing with the DecenAI platform to distribute those AI modules under the provider's policies. These AI agents communicate through the DecenAI platform using secure protocols and Application Programming Interfaces (APIs).

The term “AI module” as used herein broadly encompasses any self-contained AI algorithm, model, or computational program that can be independently deployed and executed to perform a task or subtask. A granularity of an “AI module” as used herein can vary. An AI module can be an entire AI model, or smaller functional blocks. In a non-limiting example, an AI module can be a single well-trained neural network for one step of a larger pipeline, or even a dataset or knowledge base that an AI can query.

The term “zero-knowledge proof validation service” refers to any component or components that verify conditions via zero-knowledge proof techniques, and the term “value chain” refers to any distributed ledger or blockchain component that records agreements and transfers value. Mention of specific technologies (like blockchain, zk-SNARKs, etc.) is for illustrative purposes; the invention is not limited to those particular implementations, as other cryptographic or distributed systems could be employed to achieve similar functionality.

The disclosed DecenAI platform enables AI agents to autonomously discover and utilize specialized AI modules on behalf of users, while ensuring the integrity of AI modules, the privacy of user data, and fair compensation for AI module providers. The disclosed DecenAI platform is particularly well-suited for critical and sensitive applications like healthcare diagnostics, where it can facilitate advanced analyses (e.g., genomic testing, medical image interpretation) in a secure, distributed manner. However, the disclosed architecture is broadly applicable to any domain that requires secure collaboration between data owners and AI expertise, including finance, legal, defense, and the like. By decentralizing the AI capabilities and embedding trust at every level (data, model, and transaction), the disclosed DecenAI platform heralds a new paradigm of AI deployment that empowers users with adaptable, privacy-preserving intelligence on demand.

The disclosed DecenAI platform includes a decentralized AI architecture with modular knowledge modules. The disclosed DecenAI platform breaks down AI capabilities into discrete knowledge modules that can be registered, discovered, and invoked on demand. In a non-limiting example, knowledge modules as described herein can include AI modules including specialized neural network models or algorithms for particular tasks. AI agents operating on behalf of users can dynamically acquire new skills or models by fetching these modules from a distributed Knowledge Module Registry, rather than relying on a single monolithic model. This modular approach allows highly specialized functions to be developed and maintained independently, then shared securely through the disclosed DecenAI platform. Because all AI modules adhere to a common structural standard, they can be easily integrated and orchestrated across different AI agents, facilitating a rich ecosystem of reusable AI capabilities.

To ensure trust and security, the disclosed DecenAI platform incorporates the “System And Method For Architectural Integrity Assurance In Neural Networks” (Tinsor) framework described in U.S. patent application Ser. No. 18/893,496, incorporated herein by reference. The Tinsor framework enforces that every knowledge module's internal structure (e.g., its neural network graph and tensor parameters) conforms to predefined security and format standards. In practice, this framework can be realized as a set of rules, software libraries, and validation tools that AI module providers and a user's AI agents both utilize.

When an AI module is created and uploaded to the DecenAI platform (or when it is first registered in a knowledge module registry), Tinsor tools generate a reference structural blueprint or fingerprint of the AI module's neural network graph that is stored in the knowledge module registry. In a non-limiting example, this can include hashing the sequence of layer types, the dimensional parameters of each tensor, a checksum of the model's weights, and/or other critical parameters. The Tinsor framework can enforce structural standards as a prerequisite for registry listing. In a non-limiting example, the Tinsor framework can enforce a rule requiring that an AI model file is in a particular format (such as an open neural network exchange format) and includes metadata describing its architecture. When an AI module is later retrieved for use, a Tinsor validation routine is invoked to recompute a fingerprint and ensure it matches the original reference fingerprint, thus confirming that the AI module has not been tampered with or corrupted. If any discrepancy is found, the DecenAI platform will reject the AI module, and the incident can be flagged for security review. Through this mechanism, the DecenAI platform ensures that only trusted, verified AI modules are executed.

The Tinsor framework not only hardens security but also standardizes AI module design, enabling seamless exchange and reuse of AI modules. By requiring a common representation standard, Tinsor makes it easier for different AI agents to understand and run AI modules from diverse providers without compatibility issues. Tinsor accordingly encourages a more universal standard of AI model formats across the industry, which greatly improves interoperability. AI developers would naturally produce AI modules in the standard format, making them plug-and-play across different agents and applications.

The disclosed DecenAI platform includes a knowledge module registry that serves as a decentralized database, ledger, directory, or library that maintains entries of each available AI module on the DecenAI platform. Each AI module can provide a distinct functional capability. The knowledge module registry is configured to store and index metadata about each AI module's capabilities, requirements, and trust certifications. Each entry in the knowledge module registry can include information such as, but not limited to, an AI module identifier, a description of its function or expertise area, the identity of the AI module's provider or creator including, in a non-limiting example, the AI module provider's network address, version information, compatibility requirements (e.g., what input data format is needed, what other AI modules it may depend on), and usage availability or cost terms. It is contemplated that the knowledge module registry can be implemented in a distributed manner to avoid any central point of failure or control, aligning with the decentralized nature of the disclosed DecenAI platform. Further, implementing the knowledge module registry in a distributed matter ensures that it is highly available and tamper-resistant. Tamper-resistance as used herein refers to the knowledge module registry being unable to be altered undetected. For example, the disclosed knowledge module registry is tamper-resistance as no single malicious actor would be able alter the list of available AI modules undetected. For example, the knowledge module registry could be built atop a blockchain, a distributed hash table, or another peer-to-peer network.

The knowledge module registry enables an AI agent to efficiently locate and retrieve one or more specific skills or AI models needed for a given task. AI agents querying the registry can do so by specifying the task or capability they need. The knowledge module registry will then return references to one or more AI modules that match the request. It is contemplated that AI modules in the registry can be digitally signed by their providers, and the registry can store these signatures or hashes, which are later used by the Tinsor security framework to verify integrity.

To uphold privacy and compliance without sacrificing verification, the DecenAI platform incorporates a Zero-Knowledge Proof (ZKP) validation facility. The ZKP validation facility allows the DecenAI platform to verify sensitive conditions or credentials without revealing the underlying sensitive data. The ZKP validation may be implemented using known cryptographic protocols that allow one party to prove to another that a certain statement is true without revealing any additional information beyond the truth of the statement. In the context of the DecenAI platform, the ZKP validation facility is configured to validate several types of statements. A non-limiting example of a type of statement that can require verification is user authorization or relationship proofs (e.g., verification of doctor-patient relationships). Another non-limiting example of a type of statement that can require verification is AI module usage proofs (e.g., proving that “Agent A executed Module X on Data Y exactly once” could be required to satisfy a usage contract). To enable such ZKPs, the Decen AI platform can employ cryptographic techniques like zk-SNARKs (zero-knowledge Succinct Non-interactive ARguments of Knowledge), zk-STARK (zero-knowledge Scalable Transparent ARgument of Knowledge), bulletproof, an interactive zero-knowledge proof, or other efficient ZKP schemes.

The ZKP validation facility can run on dedicated nodes (e.g., a decentralized network of verifiers for robustness) that can verify proofs submitted by AI agents. When an AI agent needs to demonstrate a condition, it generates a proof using secret inputs (such as a private credential or a cryptographic hash of data) and a public statement to be verified (e.g., “I am an authorized doctor for patient with ID hash H”). The ZKP validation facility checks this proof. If the proof is valid, the ZKP validation facility logs or signals the approval without ever learning the underlying secret information. This dramatically increases user confidence and enables compliance with privacy regulations, since it allows enforcement of rules (like “only certain people can access certain data” or “the model was used only as permitted”) in a mathematically ironclad way without exposing personal data or AI model internals. This design ensures that privacy is preserved by default, and trust is established through cryptography rather than through limiting access or manual auditing.

The disclosed platform further comprises a value chain facility, which is an economic value transfer system. The value chain facility acts as a transactional knowledge module, underpinning economic interactions on the DecenAI platform. The value chain facility is a ledger built on blockchain or distributed ledger technology (hereinafter referred to as a “blockchain ledger”). Preferably, the blockchain ledger is operated by stakeholders of the DecenAI platform (e.g., major healthcare institutions and AI providers). Alternatively, the blockchain ledger can be a smart-contract-capable public blockchain.

Each AI module on the knowledge module registry can be associated with a smart contract template on the blockchain ledger, defining how it can be rented or utilized. The smart contract specifies the terms of use (e.g., the permitted duration of use, cost or fee for the AI module, and any other usage constraints) of the particular AI module. When a user's AI agent requests an AI module, a fresh instance of the smart contract is generated to govern that particular transaction. This instance of the smart contract records the identities (or pseudonymous addresses) of the parties, the AI module involved, and the agreed terms of use (e.g., usage limits and price). The instance of the smart contract may also include clauses for automatic verification. In a non-limiting example, the instance of the smart contract can be programmed to accept a certain ZKP from the user's AI agent as evidence of AI module execution, and in response trigger a payment. Payments on the blockchain ledger can, in a non-limiting example, be made in cryptocurrency tokens native to the DecenAI platform or via integration with traditional payment systems.

The blockchain ledger securely records the instance of the smart contract and, upon fulfillment of the instance of the smart contract's terms (for example, when the AI module has been executed as agreed), automatically and autonomously self-executes. Execution of the instance of the smart contract can include, but is not limited to, handling transfer of payment or reward to the AI module's provider (e.g., locking a payment amount from a user in escrow when an AI module is delivered and releasing the payment from the escrow account to the provider's account once the AI module is used). Automatic and autonomous self-execution of the instance of the smart contract removes the need for intermediaries and ensures that providers are paid promptly and fairly for actual usage of their AI modules. Conversely, if the instance of the smart contract's terms are not met (e.g., proof of proper use is not provided, the AI module was not used within allowed time, or another expiration condition is met), execution of the smart contract can include refunding the user, disabling or deleting the AI module, or otherwise enforcing penalties as defined in the instance of the smart contract, thereby protecting all parties.

Because the blockchain ledger is distributed leveraging blockchain's immutable ledger and automation features, all transactions are transparent to the relevant participants and cryptographically verifiable, building an audit trail of AI module usage. Participants can verify that payments are made fairly and that AI module usage adheres to contractually agreed terms. Use of the blockchain ledger also incentivizes contributions to the DecenAI platform by ensuring that developers of valuable AI modules are duly compensated in a tamper-proof manner.

In the architecture of the disclosed DecenAI platform, data does not need to be sent to a central server for processing. Instead, the AI modules are brought to the data. A user's local AI agent can execute the acquired knowledge module directly on the user's device or local environment where the sensitive data resides. This privacy-by-design approach means personal data (e.g., a patient's genomic sequence or a client's financial records) remains local and under the user's control during processing. Only the results (or encrypted proofs of the results) are shared back to the requesting party. By avoiding large, centralized data stores, the disclosed DecenAI platform minimizes exposure to data breaches and complies more easily with data localization laws, since data can be processed in-place or within approved jurisdictions. Additionally, any verification of the process (via the ZKP facility) or economic transaction (via the blockchain ledger 140) occurs without divulging the data itself, preserving confidentiality end-to-end.

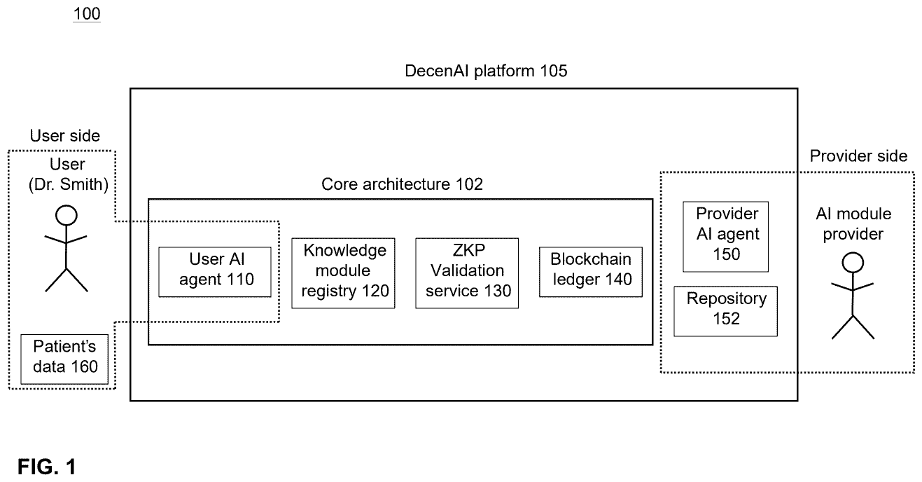

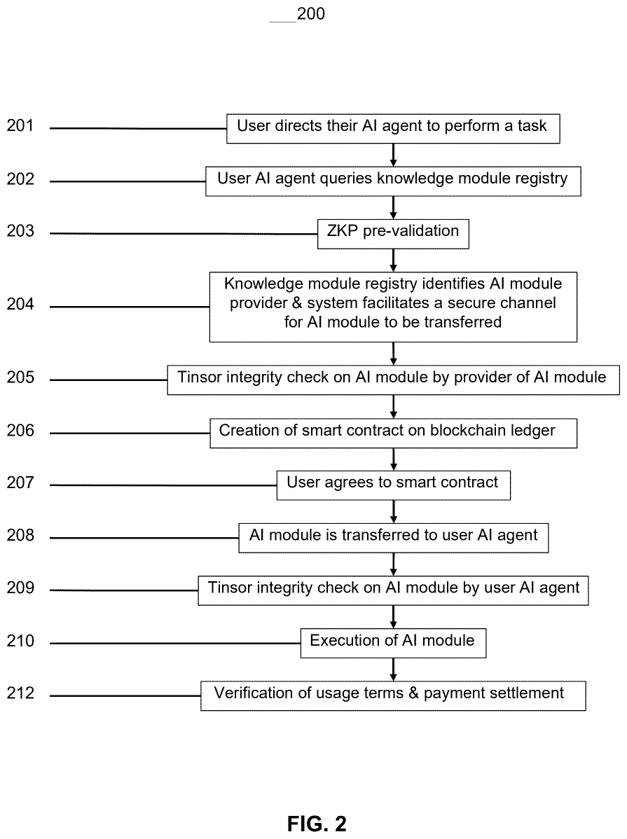

FIGS. 1-2 illustrate an exemplary use case of the disclosed DecenAI platform applied to a genomic testing scenario for medical diagnostics. FIG. 1 illustrates a system 100 including a DecenAI platform 105 in which a user, Dr. Smith (a physician), utilizes her personal AI agent 110, to conduct a genetic analysis for her patient, John. A core architecture 102 of the system 100 includes Dr. Smith's AI agent 110 (on the user side), a knowledge module registry 120, a ZKP validation service 130, a blockchain ledger 140. The system 100 also includes a testing service's AI agent 150 operated by an AI module provider on the service provider side. John's genomic data 160 is stored in a secure location accessible to Dr. Smith's AI agent 110 (e.g., in a hospital database or the patient's local data source). FIG. 2 illustrates an exemplary method 200 of using the system 100 of FIG. 1.

At block 201, a user directs their AI agent to perform a task or an AI agent autonomously determines that a task needs to be done on sensitive input data. In the illustrated example of FIG. 1, Dr. Smith instructs her AI agent 110 to perform a specialized genomic test on John's genomic data 160, aiming to identify specific disease markers.

At block 202, the user's AI agent queries the knowledge module registry 120 for an appropriate AI module capable of performing the task. In the illustrated example of FIG. 1, Dr. Smith's AI agent 110 issues a query to the knowledge module registry 120 to find an AI module capable of performing the required genomic test. The knowledge module registry 120, being a repository of numerous AI modules, searches its index for an AI module that matches the request.

At block 203, a pre-validation occurs using a ZKP validation service. The pre-validation can include the user's AI agent obtaining a credential or consent token from a user or from a third-party authority and encoding said credential or consent in the ZKP. Pre-validation criteria can include, but are not limited to, authenticating the requesting user's permissions, ensuring any data usage is approved, and confirming that the context of request complies with the AI module's usage policies. For example, some AI modules can only be licensed for certain purposes or data types. If the ZKP validation service determines that the request does not meet the necessary privacy and/or compliance conditions, the request is denied and the method 200 is completed and cannot proceed on. In the illustrated non-limiting example of FIG. 1, Dr. Smith's AI agent 110 engages with the ZKP validation service 130 to authenticate the usage context of the AI module containing the genomic test. Specifically, the DecenAI platform 105 needs to verify that Dr. Smith is authorized to run this sensitive test on John's genomic data 160. In the illustrated example, the ZKP validation service 130 is configured to confirm the doctor-patient relationship between Dr. Smith and John and confirm John's consent without exposing any of John's genomic data 160. Through a cryptographic ZKP mechanism, Dr. Smith's AI agent 110 proves to the ZKP validation service 130 that Dr. Smith (as a licensed physician) has a legitimate relationship with John (the patient) and proper authorization, all without revealing John's identity or health details to the DecenAI platform 105. Only upon successful verification does the ZKP validation service 130 signal to the knowledge module registry 120 that the request meets the necessary privacy and compliance conditions.

At block 204, the DecenAI platform identifies a provider and facilitates a secure channel for the AI module to be provided. Particularly, the knowledge module registry 120 can obtain an identifier of a selected AI module and a network address of a provider agent offering the selected AI module. In the illustrated example of FIG. 1, the knowledge module registry 120 locates an appropriate AI module and routes the request to the testing service's AI agent 150, which is an autonomous agent representing the AI module's provider. In the illustrated example, the AI module containing the genomic test is provided by a specialized testing service (e.g., a company or research institution that developed a proprietary AI module for genomic testing). The testing service's AI agent 150 is responsible for delivering the requested AI module under the correct conditions.

At block 205, the Tinsor framework is invoked by the AI module's provider to package and/or verify the AI module's integrity at its source. In the illustrated example of FIG. 1, the testing service ensures the AI module's integrity and readiness. In particular, the testing service's AI agent 150 applies the Tinsor security protocols to the AI module including the genomic test. This means the testing service's AI agent 150 checks that the AI module conforms to the required architectural integrity standards (e.g., correct structural signature, no tampering detected in its layers or weights). If the AI module was stored in a repository 152, the testing service's AI agent retrieves it and validates its structural hash or signature as defined by the Tinsor framework. By performing this Tinsor integrity check, the AI module's provider is assured that the AI module is secure and has not been altered, thereby preserving the trustworthiness of the diagnostic process.

At block 206, once the AI module's integrity is confirmed, a transactional aspect is initiated by a smart contract sub-system. Particularly, an instance of a smart contract governing usage of the AI module is created on the blockchain ledger outlining the terms of this provisioning. In the illustrated non-limiting example of FIG. 1, the testing service's AI agent 150, in communication with the blockchain ledger 140, proposes a smart contract regarding the AI module's usage. This smart contract, represented on the blockchain ledger 140, specifies terms and/or conditions for using the AI module. Non-limiting examples of terms for using the AI module can include a price or fee to be paid to the AI module's provider, the duration or number of uses allowed (e.g., a one-time analysis within a 24-hour window, or any other combination of duration and number of uses), confidentiality obligations, or expiration of the AI module after use. The terms and/or conditions of the smart contract are sent to Dr. Smith's AI agent 110 for review.

At block 207, the user agrees to the smart contract. If the user or the user's AI agent acting on behalf of the user does not agree to the terms and/or conditions of the smart contract, the method 200 is completed and cannot proceed on. In the illustrated example of FIG. 1, Dr. Smith's AI agent 110 (on behalf of Dr. Smith) evaluates the terms/conditions of the smart contract. If Dr. Smith's AI agent agrees to the terms and/or conditions of the smart contract and signals acceptance of the smart contract. Upon mutual agreement, the smart contract is recorded on the blockchain ledger 140. Execution of the smart contract by Dr. Smith and the testing service results in a binding, transparent agreement that the AI module including the genomic test can be used under the specified conditions, and that payment will be made accordingly.

At block 208, with the smart contract in place, the AI module is transferred to the user's AI agent. The AI module can be transmitted securely (for instance, encrypted in transit). In the illustrated example of FIG. 1, the testing service's AI agent 150 delivers the AI module including the genomic test to Dr. Smith's AI agent 110.

At block 209, upon the user's receipt of the AI module, the user's AI agent (or the DecenAI platform on its behalf) validates the AI module against the Tinsor standards to ensure nothing was corrupted in transit and that the AI module received is the correct authorized AI module. Cryptographic checksums or proofs embedded by the provider and understood by the Tinsor protocol can be used. In the illustrated example of FIG. 1, when Dr. Smith's AI agent 110 receives the AI module, the DecenAI platform 105 performs an additional validation. Particularly, Dr. Smith's AI agent 110 invokes the ZKP validation service 130 once more to verify the AI module's authenticity and integrity before execution. This double-check uses ZKP techniques to confirm that the AI module received is indeed the certified AI module from the provider and that the AI module has not been altered or swapped. This double-check proves, for example, that the AI module received by Dr. Smith that it meets the Tinsor structural criteria without revealing the proprietary details of the AI module's algorithm to the ZKP validation service 130. This step ensures end-to-end that the AI module received by Dr. Smith is legitimate and safe to run in Dr. Smith's environment.

At block 210, following the safeguards of blocks 201-209, the user's AI agent executes the AI module on the local input data provided by the user. During execution, the AI module may run in an instance of a secure, isolated execution runtime environment such as a sandboxed environment for additional security, and the AI module may be restricted (by the user AI agent or by the DecenAI platform) from exporting the raw data or any artifacts except the intended results. This ensures that even the AI module itself cannot exfiltrate sensitive data, aligning with the principle that data remains local to the user. An output generated by the AI module is released to the user (or to whatever entity requested the task) only after the execution of the AI module is complete and the AI module has been terminated or put in a safe state. The user can then take further actions based on the output. The AI module can be disabled or deleted/destroyed after execution if agreed upon in the smart contract (e.g., the AI module can be disabled or deleted from the user side after the agreed upon number of uses of the AI module is reached). The instance of the secure, isolated execution runtime environment can also be destroyed following execution of the AI module by the user, thereby preventing retention of the AI module on the user side beyond the terms agreed upon in the smart contract. In the illustrated example of FIG. 1, Dr. Smith's AI agent 110 is now equipped with the authorized AI module including the genomic test. Dr. Smith's AI agent 110 retrieves John's genomic data 160 (which may be stored in an electronic health record database or another secure data source to which Dr. Smith has access), executes the genomic test on John's genomic data 160, and provides results of the genomic test to Dr. Smith. The analysis is performed locally by Dr. Smith's AI agent 110 on John's genomic data 160, meaning the sensitive raw data never leaves Dr. Smith's side. The AI module processes the genomic sequence to detect any disease markers or genetic anomalies of interest within John's genomic data 160. Once the computation is complete, Dr. Smith's AI agent 110 obtains the diagnostic results. The diagnostic results indicate whether specific genetic markers associated with potential disorders were found, thereby providing Dr. Smith with critical insights into John's condition. Dr. Smith's AI agent 110 then securely delivers the results back to Dr. Smith (for example, displaying them to Dr. Smith in an interface or application) in a format that Dr. Smith can review and interpret.

At block 212, the method 200 concludes with verification and payment settlement. After execution of the AI module at block 210, the user's AI agent and the DecenAI platform can produce a proof of usage (if required by the instance of the smart contract) to confirm that the AI module was used as intended. The DecenAI platform can validate this proof via the ZKP validation service, then transmit the ZKP to the blockchain ledger. The transmission of the ZKP to the blockchain ledger triggers the blockchain ledger to transfer payment from the user's account to the provider's account. The knowledge module registry entry for the AI module can also be updated to record a usage event for reputation or auditing purposes (e.g., increment a usage counter or record feedback). If the instance of the smart contract specified a limited-time use or single-use license, the user's AI agent will securely dispose of the AI module after use (e.g., wiping it from memory or storage) to honor the terms of the instance of the smart contract and prevent any unlicensed reuse. This can also be enforced by the AI module itself. For example, the AI module can be packaged to self-delete after execution and verified later if needed. In the illustrated example of FIG. 1, the terms and/or conditions of the smart contract on the blockchain ledger 140 are enforced. Namely, the DecenAI platform 105 triggers the ZKP validation service 130 to verify that the AI module including the genomic test was used legitimately on John's data as per the smart contract. In a non-limiting example, this may involve Dr. Smith's AI agent 110 providing a ZKP that it indeed ran the AI module on the authorized data (John's data) and only within the scope allowed (e.g., one-time use). Because the ZKP validation service does not leak any patient data or AI module internals, it preserves privacy while proving compliance. Once this usage verification is confirmed, the smart contract automatically releases payment from Dr. Smith's side to the testing service provider. Specifically, the blockchain ledger 140 executes the transfer of the agreed fee to the testing service's AI agent 150 (or to the provider's cryptocurrency wallet address associated with the AI module), completing the economic transaction. The testing service's AI agent 150 receives the payment as compensation for providing the AI module, and the smart contract is thereby fulfilled and closed.

Through this example illustrated in FIGS. 1-2, one can appreciate how the various components of the DecenAI platform interact to enable a secure and private AI-driven solution. All parties' needs are satisfied: Dr. Smith obtains a highly specialized AI analysis for her patient without compromising patient privacy (John's genomic data 160 was never exposed to the provider of the AI module or any central server), John's sensitive information remains confidential, the AI module provider is compensated fairly and retains control over their intellectual property (the proprietary algorithm within the AI module was used only under agreed terms and not exposed), and the DecenAI platform 105 ensured compliance and trust at each block via cryptographic validation. This genomic testing use case is just one example; the same architecture can facilitate numerous other critical tasks, from medical imaging analysis to financial fraud detection, where secure module exchange and privacy preservation are paramount.

It should be noted that various modifications and alternative use cases will be apparent to those skilled in the art without departing from the scope of the claims. For instance, while the example of FIG. 1 is in a healthcare context, it is contemplated that the DecenAI platform can be applied to other sensitive data scenarios including, but not limited to a financial auditor's agent fetching a fraud detection model to run on private transaction data, or an intelligence analyst's agent obtaining a specialized image recognition module to run on confidential satellite imagery. In each use case, the same components, namely the knowledge module registry, Tinsor integrity checks, ZKP validations for permissions, and a blockchain contract for payment or access logging can be used in concert to enable completion of a task securely. The Tinsor framework could be extended to cover not just neural network models but also other forms of AI assets (e.g., rule-based systems or statistical models) by defining appropriate structural standards for each type. The value chain transactions could also involve composite services (e.g., paying not just the model provider but also data providers or other contributors, by splitting the transaction among multiple parties as encoded in the smart contract).

This written description uses examples to describe aspects of the disclosure described herein, including the best mode, and also to enable any person skilled in the art to practice aspects of the disclosure, including making and using any devices or systems and performing any incorporated methods. The patentable scope of aspects of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A decentralized artificial intelligence system, comprising:
a knowledge module registry configured to store and index a plurality of entries, each entry corresponding with a respective AI module, each AI module providing a distinct functional capability;
a security framework enforcing an architectural integrity policy for the plurality of AI modules, wherein the security framework verifies that each AI module of the plurality of AI modules conforms to a predefined structural standard to detect tampering or unauthorized modifications;
a zero-knowledge proof validation facility configured to verify one or more conditions associated with use of an AI module of the plurality of AI modules or access to data by generating or checking cryptographic proofs, wherein the one or more conditions are configured to be verified by the zero-knowledge proof validation facility without disclosing underlying sensitive data or internal details of any of the plurality of AI modules to the zero-knowledge proof validation facility;
a distributed ledger configured to record usage transactions and smart contracts associated with the plurality of AI modules, the distributed ledger configured to implement a blockchain-based value transfer system for exchanging payments or credits between participants; and
a plurality of agent computing nodes each communicatively coupled to the knowledge module registry and the distributed ledger, the plurality of agent computing nodes comprising:
a client agent node configured to request a selected AI module of the plurality of AI modules stored on the knowledge module registry on behalf of a user and to execute the selected AI module on a local input data to generate an output result; and
a provider agent node configured to host or deliver the selected AI module in response to the request, wherein the provider agent node cooperates with the zero-knowledge proof validation facility to ensure that predetermined usage conditions of the selected AI module are satisfied before or during delivery of the selected AI module, and wherein the provider agent node interacts with the distributed ledger to establish a smart contract governing usage of the selected AI module and to receive compensation via the blockchain-based value transfer system upon verified completion of usage of the selected AI module.

2. The decentralized artificial intelligence system of claim 1, wherein:
the knowledge module registry is implemented as a decentralized repository on a peer-to-peer network or blockchain ledger, such that plurality of entries stored in the knowledge module registry are tamper-resistant and globally accessible; and
wherein each entry in the knowledge module registry includes metadata describing functionality, version, provider identity, and usage terms of a respective AI module of the plurality of AI modules.

3. The decentralized artificial intelligence system of claim 1, wherein:
at a time of registration in the knowledge module registry, a reference structural fingerprint of each AI module of the plurality of AI modules is stored in the knowledge module registry;
the security framework enforcing the architectural integrity for the selected AI module comprises a set of structural verification rules for neural network models;
the security framework generates a structural fingerprint for each AI module of the plurality of AI modules by analyzing its computational graph or layers; and
the security framework is configured to validate the selected AI module by comparing its structural fingerprint at runtime to its reference fingerprint to detect any discrepancy.

4. The decentralized artificial intelligence system of claim 1, wherein the zero-knowledge proof validation facility is further configured to authenticate an authorization of the user or relationship of the user to a data subject by verifying a zero-knowledge proof that encodes said relationship, whereby the decentralized artificial intelligence system is configured to confirm that the user has permission to use the selected AI module on the local input data without the decentralized artificial intelligence system learning an identity of the data subject or a content of the local input data.

5. The decentralized artificial intelligence system of claim 1, wherein the zero-knowledge proof validation facility is further configured to verify integrity and proper usage of the selected AI module by checking a cryptographic proof generated by the client agent node after execution of the selected AI module, the cryptographic proof attesting that the execution occurred in compliance with terms of the smart contract without revealing the local input data or the output result.

6. The decentralized artificial intelligence system of claim 1, wherein:
the distributed ledger comprises a smart contract subsystem that automatically executes payment transactions;
the smart contract subsystem locks a payment amount in escrow when the selected AI module is delivered to the client agent node and releases payment to the provider agent node upon receiving confirmation from the zero-knowledge proof validation facility that the selected AI module was used in accordance with agreed terms.

7. The decentralized artificial intelligence system of claim 1, wherein the client agent node is configured to maintain the local input data locally and to execute the selected AI module in a secure local environment, such that any raw input data is not transmitted to the provider agent node or any central server, thereby preserving privacy of the local input data.

8. A computer-implemented method for secure deployment and execution of an AI module in a decentralized network, comprising:
(a) receiving, by a client AI agent executing on a user device on behalf of a user, a request to perform a task on sensitive input data;
(b) querying a knowledge module registry for an AI module capable of performing the task, and obtaining an identifier of a selected AI module and a network address of a provider agent offering the selected AI module;
(c) prior to the client AI agent obtaining the selected AI module, verifying at least one usage condition selected from the group consisting of: the user or client AI agent having authorization to use the selected AI module, the user having a valid relationship or credential required for the task, or the sensitive input data meeting criteria required by the AI module by providing a first zero-knowledge proof to a validation service, wherein the first zero-knowledge proof attests to the at least one usage condition, and wherein the first zero-knowledge proof is verified by the validation service without revealing underlying details of the authorization, relationship, or sensitive input data;
(d) establishing a smart contract on a blockchain ledger between the client AI agent and the provider agent, the smart contract specifying terms for usage of the selected AI module including at least a permitted usage scope including at least a usage count and a payment amount;
(e) transferring the selected AI module from the provider agent to the client AI agent in accordance with the smart contract including the client AI agent receiving a program or model data package representing the AI module;
(f) the client AI agent validating by a security framework that the selected AI module transferred to the client AI agent conforms to an expected architecture or integrity constraint stored for the selected AI module, and aborting the method if the validation fails;
(g) upon successful validation, executing the AI module by the client AI agent on the sensitive input data locally on the user device to produce an output result, wherein the sensitive input data remains stored locally during execution of the AI module;
(h) generating a second zero-knowledge proof of proper usage of the selected AI module, wherein the second zero-knowledge proof confirms compliance with the permitted usage scope defined in the smart contract, including at least one of: confirmation that the AI module was executed only on the sensitive input data, confirmation that the AI module was not used more times than the usage count, or confirmation that an integrity of the AI module was maintained throughout execution of the AI module; and
(i) transmitting the second zero-knowledge proof generated in step (h) to the blockchain ledger, such that the smart contract on the blockchain ledger automatically triggers a payment transfer of the payment amount from an account associated with the client AI agent to an account associated with the provider agent upon successful verification of the second zero-knowledge proof.

9. The method of claim 8, wherein in step (c) providing the first zero-knowledge proof includes the client AI agent obtaining a credential or consent token from a user or from a third-party authority and encoding said credential or consent in the first zero-knowledge proof to demonstrate that the user has rights to use the sensitive input data with the selected AI module, whereby the validation service enforces data privacy regulations by not revealing any actual identity or contents of the input data during the verification.

10. The method of claim 8, wherein the smart contract in step (d) specifies a time window or expiration condition for use of the AI module, and wherein the method further comprises the client AI agent disabling or deleting the AI module after step (g) if the time window has elapsed or the usage count is exhausted, thereby preventing unauthorized reuse of the AI module beyond the terms of the smart contract.

11. The method of claim 8, wherein the security framework in step (f) computes a cryptographic hash or checksum of a structural representation of the AI module including at least its architecture topology or weight parameters and compares it to a reference hash obtained from the knowledge module registry or the provider agent, wherein matching hashes indicate that the AI module is authentic and has not been altered.

12. The method of claim 8, wherein the execution in step (g) is carried out in an isolated execution environment on the user device to prevent the AI module from transmitting the sensitive input data or the output result externally, and wherein the output result is released to the user only after the execution of the AI module is complete and the AI module has been terminated or put in a safe state.

13. The method of claim 8, wherein the second zero-knowledge proof generated in step (h) is constructed such that it does not reveal the output result of the AI module, whereby even the provider agent or any other party reviewing the second zero-knowledge proof or the blockchain ledger cannot learn the output result.

14. A non-transitory computer-readable storage medium storing instructions that, when executed by one or more processors of a computing system, cause the computing system to perform a method comprising:
querying a decentralized registry service for a description of an AI module configured to perform a specified data processing task;
receiving metadata from the decentralized registry service identifying a candidate AI module and a provider service capable of providing the candidate AI module;
obtaining verification, via a zero-knowledge proof protocol, that a set of access conditions for using the candidate AI module are satisfied by an entity associated with the computing system, wherein the access conditions are verified without revealing secret data associated with the entity;
sending a request to the provider service for the candidate AI module and negotiating a usage agreement, wherein the usage agreement is recorded as a smart contract for a transaction on a blockchain ledger and includes conditions including at least a payment term and a usage policy term;
in response to the usage agreement, receiving a data package representing the candidate AI module;
validating the data package by checking that it adheres to a predetermined structural template or security signature corresponding to the candidate AI module;
executing the AI module on an input dataset accessible to the computing system to generate a result, wherein the input dataset is not transmitted to the provider service during execution; and
upon completion of execution, transmitting a signal or proof to the blockchain ledger indicating that the usage policy term of the usage agreement has been met, whereby the smart contract triggers an automated transfer of a payment amount to the provider service.

15. The non-transitory computer-readable storage medium of claim 14, wherein the instructions further cause the computing system, prior to executing the AI module, to instantiate a secure runtime environment for the AI module, and to destroy or deactivate the secure runtime environment after obtaining the result, thereby preventing retention of the AI module on the computing system beyond the usage policy term.

16. The non-transitory computer-readable storage medium of claim 14, wherein the predetermined structural template comprises one or more of: a required file format, a specific neural network architecture schema, a digital signature from a trusted authority, or embedded meta-data tags, and wherein validating the data package comprises ensuring the data package matches the template and verifying the digital signature to confirm authenticity of the AI module.

17. The non-transitory computer-readable storage medium of claim 14, wherein the zero-knowledge proof protocol used to obtain verification of access conditions employs a cryptographic proof selected from the group consisting of: a zk-SNARK, a zk-STARK, a bulletproof, or an interactive zero-knowledge proof, and wherein the access conditions include at least one of a user credential validation, a data compliance validation, or a context authorization validation required by the provider service.

18. The non-transitory computer-readable storage medium of claim 14, wherein the blockchain ledger is configured to log a record of the transaction including a hash of the transmitted signal or proof and references to the AI module and the input dataset in hashed or anonymized form, such that an audit trail is created that stakeholders can later review to confirm that the specified data processing task was performed under the conditions of the smart contract without revealing any underlying data or the result.
